(19) United States
(12) Patent Application Publication
(10) Pub. No.: US 2024/0122620 A1
(43) Pub. Date: Apr. 18, 2024

(54) NEEDLE GUIDE

(71) Applicant: Medical Templates AG, EGG BEI ZÜRICH (CH)

(72) Inventors: Stephan WETZEL, EGG BEI ZÜRICH (CH); Rafael MOKRY-HOSTETTLER, Merelbeke (BE)

(21) Appl. No.: 18/546,008

(22) PCT Filed: Feb. 11, 2022
(86) PCT No.: PCT/EP2022/053321

§ 371 Date: Aug. 10, 2023

(30) Foreign Application Priority Data
Feb. 23, 2021 (DE) 10 2021 104 222.4

(51) Int. Cl. A61B 17/34

(52) U.S. Cl. CPC A61B 17/3403; A61B 2017/3407; A61B 2017/3411

(57) ABSTRACT

The invention is a needle guide for performing intracorporeal location of a puncture needle to be percutaneously introduced into a patient, comprising an upper plate element, which face away from the patient, and a lower plate element, which faces the patient. The plate elements are rigidly spaced apart from one another and each provide through holes through which the puncture needle is guided. The invention has an upper and lower plate element which is separatable into at least two plate element parts, which, when joined together, commonly surround all of the through holes for each plate element.

### CROSS-REFERENCE TO RELATED APPLICATIONS

Reference is made to PCT/EP2022/053321 filed Feb. 11, 2022, and German Patent Application No. 10 2021 104 222.4 filed Feb. 23, 2021, which are incorporated herein by reference in their entirety.

### BACKGROUND OF THE INVENTION

### Field of the Invention

The invention relates to needle guides for intracorporeal locating of a puncture needle to be percutaneously introduced into a patient, comprising an upper plate element, which can be situated facing away from the patient, and a lower plate element, which can be situated facing the patient, with plate elements being rigidly spaced apart from one another and each providing holes through which the puncture needle is guided.

### Description of the Prior Art

Several medical situations exist, in which a controlled introducing and positioning of a puncture needle at a particular site in the body of patient is necessary. As puncture needles, in particular biopsy needles are used for the removal of tissue samples. In addition, injection needles for the controlled administering of a substance in a particular region in the interior of the body, and guiding needles for operation screws or drills come into consideration. Likewise, puncture needles are used within medical therapies for targeted stimulation. In all these applications, it is necessary to position a defined part of the puncture needle—primarily its tip—at a desired location in the interior of the body. Furthermore, a controlled introducing of the puncture needle is necessary, that is also the puncture site on the surface of the body, and the puncture channel are selected on the basis of various criteria.

When at least one of precise an introduction or positioning as possible of the puncture needle is necessary, the procedure is carried out with the aid of imaging methods. For this purpose, several introducing- and positioning devices have already been proposed. As imaging methods, primarily X-ray tomography (CT), fluoroscopy, 3D X-ray, and magnetic resonance tomography (MRT) are used.

A preferred needle guide for a precise positioning and also intracorporeal introducing of a puncture needle is described in the publication WO 2017/202590 A1. The needle guide is able to be placed on the skin surface of a patient for the purposes of needle positioning and has an upper plate element, situated facing away from the patient, and a lower plate element, situated facing the patient. Both plate elements are rigidly spaced apart from one another and each provide through holes through which a puncture needle is guided. In order to increase the positioning accuracy of the puncture needle, the individual through holes, which are present in the plate elements provide through hole contours formed in a star-shaped or respectively pillow-shaped manner, with respectively have four support site regions situated in the corners. The geometry is selected so that a puncture needle is always guided by two opposite flanks and is centered within the respective support site region. In this way, an inadvertent slipping of the puncture needle within a through hole, which is generally dimensioned significantly larger than the outer diameter of a puncture needle, is as far as possible prevented. Of course, alternative geometries are also conceivable for the configuration of the through holes. In order to increase the number of possible support site regions within a through hole, for example more than four placing possibilities can be provided for the puncture needle by, for example, the number of star-shaped corners being increased.

Although the needle guide which is explained above contributes significantly to an exact positioning and introducing of a puncture needle into the patient, the needle guide after intracorporeal positioning has taken place for particular further diagnostic or therapeutic procedures on the patient, constitutes a mechanical obstacle which can be removed from the patient by extremely careful withdrawing along the spatial extent of the extracorporeal puncture needle section. However, this procedure requires a manual extraction process which is extremely difficult to carry out, by the attending respective doctor. Already, the slightest force effects directed transversely to the puncture needle due to an incorrect withdrawal of the needle guide in the longitudinal extent of the extracorporeal puncture needle region can ultimately cause a misalignment of the intracorporeal puncture needle region. Furthermore, it can be necessary to adjust or respectively correct the needle which has been introduced intracorporeally if, for example, it is found in control images that the needle misses the advised target, for example through displacement or respectively moving of the target by respiratory excursions. In this case also a removal of the needle guide can be useful and can increase the security of the procedure.

### SUMMARY OF THE INVENTION

The invention is based on solving the problem of further developing a needle guide for intracorporeal locating of a puncture needle to be percutaneously introduced into a patient, comprising an upper plate element, which can be situated facing away from the patient, and a lower plate element, which can be situated facing the patient, with plate elements rigidly spaced apart from one another. Each plate provides through holes through which the puncture needle is guided, in such a way so that a withdrawal or respectively removal of the needle guide from an intracorporeally placed and located puncture needle is possible in a gentle manner for the patient and without the risk of misalignment of the intracorporeally located puncture needle region.

According to the invention, a needle guide for intracorporeal location of a puncture needle to be percutaneously introduced into a patient has the upper and lower plate element which each can be separated into at least two plate element parts, and in the at least two plate element parts, when joined together, commonly delimit all of the through holes for each plate element.

In a preferred embodiment, the at least two plate element parts of the upper plate element are connected to an upper frame surrounding the upper plate element. Likewise, the at least two plate element parts of the lower plate element are connected to a lower frame surrounding the lower plate element. The upper and lower frame are in addition, rigidly connected to one another via a connecting structure, which is optionally able to be separated, for example by way of a detachably fixed plug connection, so that the upper frame and lower frame are able to be separated from one another.

The at least two plate element parts are preferably formed in an interdigital finger structure, that is each plate element has finger elements, formed into a comb, so that the finger longitudinal extents are respectively oriented parallel to one another. A plurality of half of the through hole contours are arranged respectively along the finger elements, such that when the at least two plate element parts are joined together, the respective half through hole contours of two immediately adjacent finger elements respectively in pairs entirely delimit the through holes.

In a first preferred embodiment, the at least two plate element parts of the upper plate element are able to be connected to the upper frame in a detachably fixed manner, and the at least two plate element parts of the lower plate element are able to be connected to the lower frame in a detachably fixed manner. In this way, it is possible, with a frame otherwise at rest, to respectively detach the two plate element parts for each plate element from the frame and to remove them from the puncture needle separately from one another and as far as possible in a contact-free or respectively low-contact manner through respective separation. The two frames, which are rigidly connected to one another via the connecting structure, can furthermore be withdrawn from the intracorporeally located puncture needle in a contact-free manner.

A further preferred embodiment makes possible that the upper and lower frame are able to be separated into at least two frame parts. In this case, respectively the at least two plate element parts for each plate element can be fixedly connected to the respective frame parts. Of course, in this case it is also possible to configure the at least two plate element parts of the respectively upper and lower plate element in a detachably secure manner to the respectively upper and lower frame part.

Through the ability of the upper and lower frame to be separated or respectively divided, the frame parts, which are to be separated here, can be removed from the puncture needle laterally in opposite directions. In particular in cases in which the proximal end of the puncture needle is connected to a continuing catheter, through the separability of the upper and lower frame a laborious unthreading of the remaining frame construction along the proximally-leading catheter can also be avoided.

### BRIEF DESCRIPTION OF THE DRAWINGS

The invention is described by way of example below without restriction of the invention with the aid of example embodiments with reference to the drawings. There are shown:

FIG. 1 illustrates a needle guide as structural a unit; and

FIG. 2 illustrates an exploded view of the needle guide with individual components separated from one another.

### DETAILED DESCRIPTION OF THE INVENTION

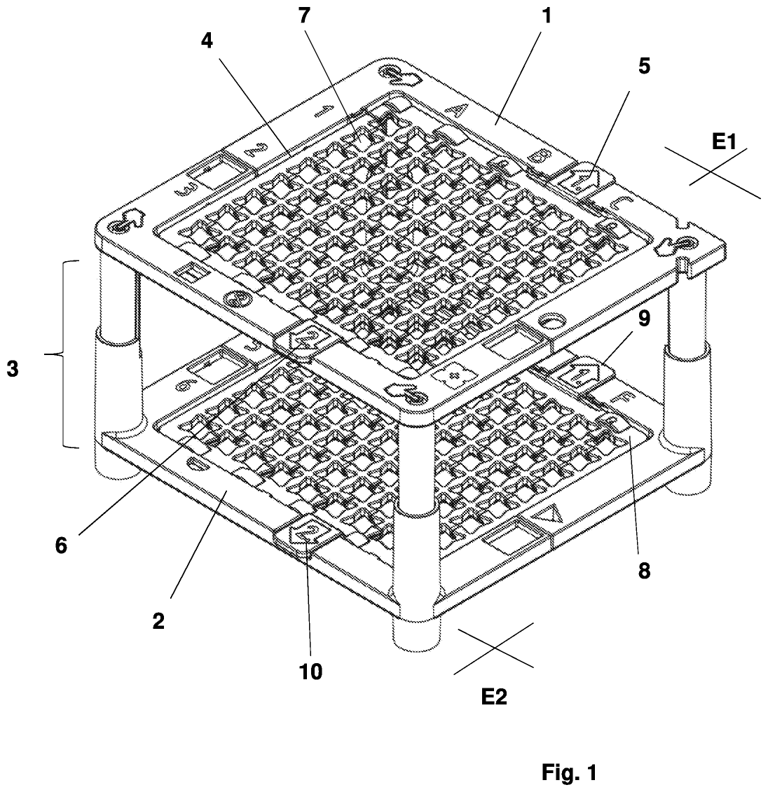

FIG. 1 shows a needle guide configured according to the invention which is a structural unit for the deployment of a positioning and introducing of a puncture needle into a patient. Neither the patient nor the puncture needle are illustrated in FIG. 1. In the illustrated example embodiment, the needle guide has an upper frame 1 and a lower frame 2, which are each configured in a square configuration. The upper frame 1 and the lower frame 2 span upper and lower frame planes E1, E2, which are arranged spatially parallel and orthogonally spaced apart from one another. The upper and lower frame 1, 2 have connecting struts 3 formed rigidly respectively at the four frame corners, connecting struts of the two frames 1, 2 which are spatially fix in a predetermined association. All four frame struts 3 together form a rigidly-connecting connecting structure in the upper and lower frames 1, 2. The upper frame 1 surrounds an upper plate element 4, which is connected to the upper frame 1 via two joining regions 5, 6. Otherwise, the upper plate element 4 is surrounded loosely by the frame 1. The upper plate element 4 has through holes 7, which are arranged in a grid-shaped manner. Each of the through holes 7 is configured in an identical manner and has a through hole contour, formed in a star configuration with respectively four corner regions. Each corner region of a through hole 7 forms a stable and spatially defined support site region for the puncture needle. The upper plate element extends in a parallel manner within the upper plate plane E1.

In the same way, the lower plate element 8 is connected to the lower frame 2 via two connecting regions 9, 10. In the same way as the upper plate element 4, the lower plate element 8 has a plurality of through holes 7 arranged in a grid-like manner.

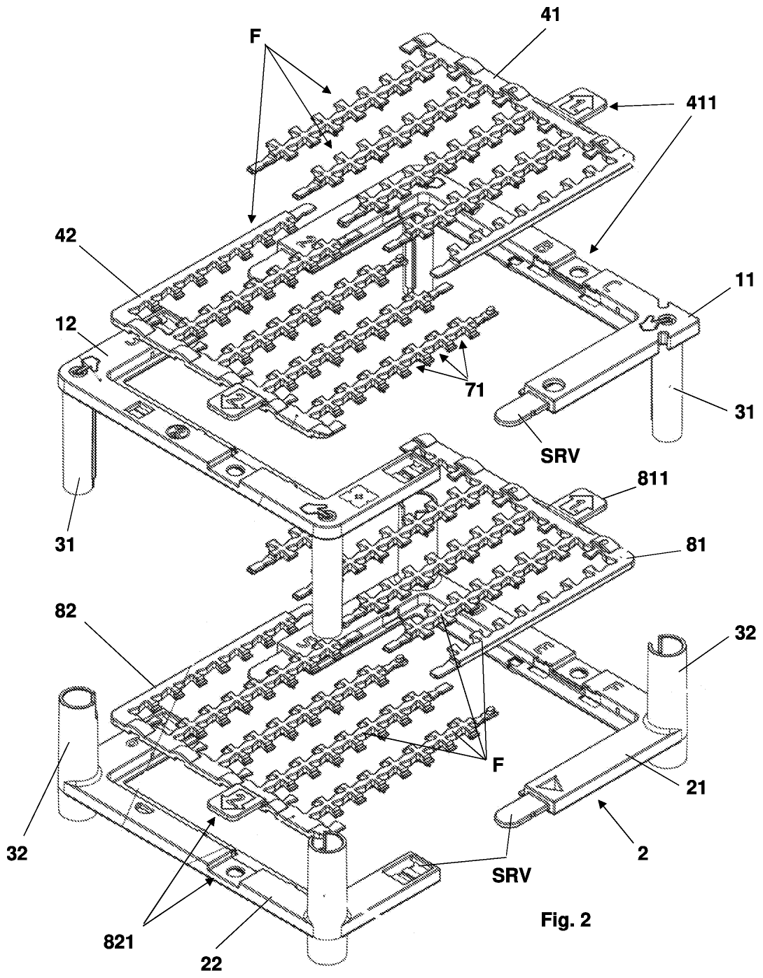

According to the invention, the upper plate element 4 is separatable into at least two plate element parts 41, 42, and the lower plate element 8 is separatable into at least two plate element parts 81, 82. In FIG. 2 respectively the upper plate element 4 and also the lower plate element 8 are shown in separated states. The upper plate element 4 and also the lower plate element 8 are composed respectively of two plate element parts 41, 42 or respectively 81, 82, which are formed in the manner of an interdigital finger structure. Each plate element part 41, 42, 81, 82 has finger elements F, formed in a comb-configuration, with finger longitudinal extents oriented respectively parallel to one another. Half-formed through hole contours 71 are arranged along the finger elements F. When the respectively half formed through hole contours 71 along the finger elements F are joined together, respectively two half formed through hole contours completely surround a through hole 7.

Each plate element 41, 42 or respectively 81, 82 is able to be connected to the upper frame 1 or respectively lower frame 2 via a detachably fixed connecting structure 411, 421, 811, 821.

Not necessarily, but in an advantageous form, the upper frame 1 is able to be separated into two upper frame parts 11, 12 via two plug detent connections SRV.

In the same, not necessarily however advantageous form, the lower frame 2 is also able to be separated into two lower frame parts 21, 22 via two plug detent connections SRV.

Likewise, not necessarily but in an advantageous form, all the connecting struts 3 are configured so as to be separable, i.e. each of the connecting struts 3 is composed of two strut segments 31, 32, which are able to be put together via a detachable plug-in mechanism.

### LIST OF REFERENCE NUMBERS

1 upper frame
11, 12 upper frame part
2 lower frame
21, 22 lower frame part
3 connecting struts
31, 32 strut segments
4 upper plate element
41, 42 upper plate element parts
411, 421 connecting structures
5 connecting region
6 connecting region
7 through hole
8 lower plate element
81, 82 upper plate element part
811, 821 connecting structure
9 connecting region
10 connecting region
71 half through hole contour
E1 upper frame plane
E2 lower frame plane
F finger element
SRV plug-in detent connection

1-8. (canceled)

9. A needle guide for intracorporeal location of a puncture needle to be percutaneously introduced into a patient, comprising an upper plate, which is situatable to face away from the patient, and a lower plate, which is situatable to face the patient, the plates being rigidly spaced apart from each other and each providing holes through which the puncture needle is guided, wherein:
the upper and lower plates are each separatable into at least two plate parts; and
when joined together, the at least two plate parts of each plate commonly define all of the holes through which the puncture needle is guided.

10. The needle guide according to claim 9, wherein:
the at least two plate parts of the upper plate are connected to an upper frame surrounding the upper plate;
the at least two plate parts of the lower plate are connected to a lower frame surrounding the lower plate; and
the upper and lower frames are rigidly connected together by a connecting structure.

11. The needle guide according to claim 9, wherein:
the at least two plate parts each include fingers.

12. The needle guide according to claim 11, wherein:
the fingers of each plate include longitudinal extensions oriented parallel to each other;
the fingers of each plate respectively include half hole contours and are arranged so that when the at least two plate parts are joined together, the half hole contours of the fingers of two immediately adjacent fingers define one of the holes.

13. The needle guide according to claim 12, wherein:
the fingers of each plate include longitudinal extensions oriented parallel to each other;
the fingers of each plate respectively include half hole contours and are arranged so that when the at least two plate parts are joined together, the half hole contours of the fingers of two immediately adjacent fingers define one of the holes.

14. The needle guide according to claim 10, wherein:
the upper and lower frames are each separatable into at least two parts.

15. The needle guide according to claim 11, wherein:
the upper and lower frames are each separatable into at least two parts.

16. The needle guide according to claim 12, wherein:
the upper and lower frames are each separatable into at least two parts.

17. The needle guide according to claim 13, wherein:
the upper and lower frames are separatable into at least two parts.

18. The needle guide according to claim 10, wherein:
at least two plate parts of the upper plate are connectably detachable to the upper frame, and the at least two plate parts of the lower plate are connectably detachable to the lower frame.

19. The needle guide according to claim 11, wherein:
at least two plate parts of the upper plate are connectably detachable to the upper frame, and the at least two plate parts of the lower plate are connectably detachable to the lower frame.

20. The needle guide according to claim 12, wherein:
at least two plate parts of the upper plate are connectably detachable to the upper frame, and the at least two plate parts of the lower plate are connectably detachable to the lower frame.

21. The needle guide according to claim 13, wherein:
at least two plate parts of the upper plate are connectably detachable to the upper frame, and the at least two plate parts of the lower plate are connectably detachable to the lower frame.

22. The needle guide according to claim 14, wherein:
at least two plate parts of the upper plate are connectably detachable to the upper frame, and the at least two plate parts of the lower plate are connectably detachable to the lower frame.

23. The needle guide according to claim 10, wherein:
the connecting structure is separable from the guide.

24. The needle guide according to claim 11, wherein:
the upper and lower frame respectively span frame planes which are oriented parallel to each other; and
the connecting structure comprises at least two connecting struts oriented orthogonal to the upper and lower frame planes.
